(19) United States
(12) Patent Application Publication
(10) Pub. No.: US 2025/0228983 A1
(43) Pub. Date: Jul. 17, 2025

(54) PROBES FOR CELLULAR SENESCENCE

(71) Applicant: UNIVERSITY OF FLORIDA RESEARCH FOUNDATION, INCORPORATED, Gainesville, FL (US)

(72) Inventors: Lina CUI, Gainesville, FL (US); Zixin CHEN, Gainesville, FL (US); Jun LIU, Gainesville, FL (US)

(21) Appl. No.: 18/849,034

(22) PCT Filed: Mar. 21, 2023
(86) PCT No.: PCT/US2023/015807

§ 371 Date: Sep. 20, 2024

Related U.S. Application Data
(60) Provisional application No. 63/321,993, filed on Mar. 21, 2022.

(51) Int. Cl. A61K 51/04; A61K 49/00

(52) U.S. Cl. CPC A61K 51/0421; A61K 49/0052; A61K 51/0497; A61K 2123/00

(57) ABSTRACT

Disclosed are imaging probes for detecting senescent cells based on quinone methide chemistry for different in vivo imaging modalities including near-infrared (NIR), positron emission tomography (PET) and magnetic resonance (MR) imaging and the methods of use thereof.

### GOVERNMENT SUPPORT CLAUSE

This invention was made with government support under R35 GM124963 awarded by the National Institutes of Health and W81XWH-17-1-0529 awarded by the U.S. Army Medical Research Acquisition Activity. The government has certain rights in the invention.

### BACKGROUND

Cellular senescence is a state where cells enter stable cell cycle arrest, which can be triggered in response to various stressors such as telomere shortening, oxidative stress, DNA damage or oncogene activation¹⁻⁴. Senescence can be either detrimental or beneficial depending on the specific circumstances. It is generally recognized that aging or chronic damaging can cause the accumulation of senescent cells, which can lead to chronic inflammation, a variety of age-related diseases⁵⁻¹¹ and cancer¹²⁻¹⁴. On the other hand, growing evidence show that senescence can also signal immune responses in response to acute damage and facilitates wound healing and tumor suppression¹⁵⁻¹⁹. The complex nature of senescence requires more comprehensive studies both in vitro and in vivo.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1. Schematic illustration of the design of self-immobilizing imaging probe via quinone methide chemistry.

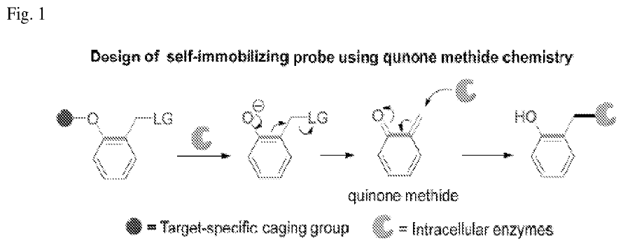

FIG. 2. General design of self-immobilizing NIR probes for imaging SA-β-Gal.

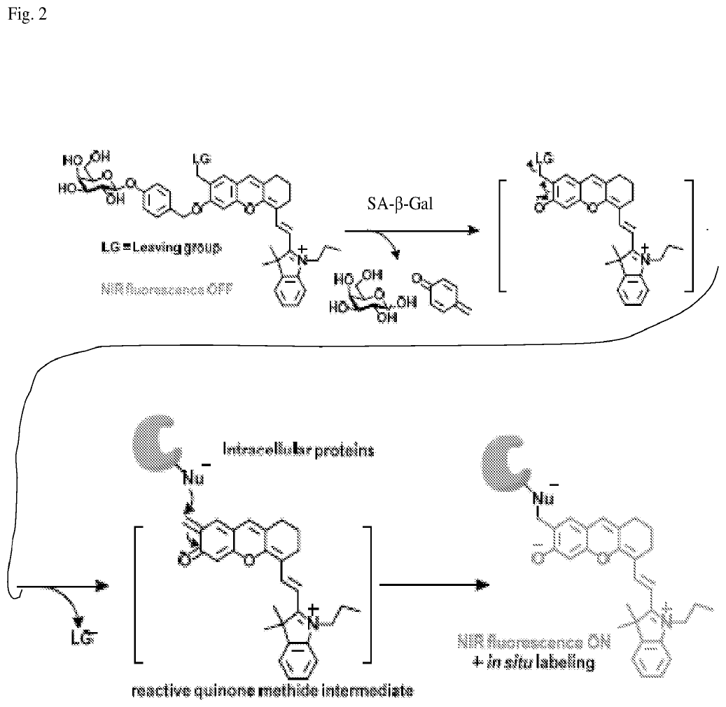

FIG. 3. General structure of new NIR SA-β-Gal probes.

FIG. 4. Compare labeling efficiency using in gel fluorescence. 10 μM of NIR-BG2, NIR-BG3 or NIR-BG4 is incubated with 10 units of β-gal and/or 50 μg of BSA in total volume of 50 μL PBS solution were incubated at 37° C. for 2 h. Each lane was loaded with 10 μL of the reaction solution.

FIG. 5. FIG. 5A: Fluorescent cell imaging of CT26. WT and CT26.CL25 cells after incubating with 10 μM of NIR-BG2 or NIR-BG3 in serum free medium for 2 hours at 37° C. Cells were fixed, permeabilized with 0.5% Triton X-100 and stained with DAPI. Scale bar: 50 km. FIG. 5B: Flow cytometry and FIG. 5C quantification of fluorescence in CT26.WT and CT26.CL25 cells after incubating with 10 μM of NIR-BG2 or NIR-BG3.

FIG. 6. FIG. 6A and FIG. 6B present general structures of PET SA-β-Gal probes.

FIG. 7. General structure of MRI SA-3-Gal probes.

FIG. 8. Synthesis of NIR-BG3 and NIR-BG4.

FIG. 9. PET imaging of mice injected with a PET SA-3-Gal probe as shown in FIG. 6, wherein R18=NOTA, and ⁶⁸Ga is the radioactive metal chelated to NOTA. Injection dose: 100 uCi; Imaging acquisition: 1h-dynamic PET imaging and twice static PET/CT scan at 2 h and 4 h post injection. Representative PET images: 1 min, 5 min, 10 min, 30 min, 1 h, 2 h and 4 h.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art.

The methods and techniques of the present invention are generally performed according to conventional methods well known in the art and as described in various general and more specific references that are cited and discussed through the present specification unless otherwise indicated.

The term “about” means plus or minus 1%, 2%, 3%, 4%, 5%, 6%, 7%, 8%, 9%, or 10% of the number to which reference is being made.

The terms “β-galactosidase”, “β-gal”, “senescence-associated β-galactosidase”, or “SABG” refer to a hydrolase enzyme that catalyzes the hydrolysis of β-galactosides into monosaccharides only in senescent cells. In senescent cells, beta-galactosidase activity is detectable at pH 6.0 whereas in normal cells, β-galactosidase is detectable at pH 4.5.

The term “contrast agent” refers to a substance used to increase the contrast of structures, tissues or fluids within the body in molecular imaging. In optical imaging, contrast agents can absorb external light and emit altered light at locations of interest. In X-rays, contrast agents enhance the radiodensity in a target tissue or structure. In MRIs, contrast agents alter the relaxation times of nuclei within body tissues thereby providing contrast in the image. In positron emission tomography (PET) or single photon emission computed tomography (SPECT) imaging, contrast is generated when contrast agents that emit positron or gamma rays accumulate in certain organs or tissues in the body. Several types of contrast agent are in use in medical imaging, and they can roughly be classified based on the imaging modalities where they are used. Most common medical contrast agents work based on positron emission tomography (PET) and magnetic resonance signal enhancement. Radioactive isotopes such as ¹⁸F, ⁶⁴Cu, ⁶⁸Ga and ⁸⁹Zr are used in commonly used in PET contrast agents. Paramagnetic metals such as gadolinium are commonly used in magnetic resonance imaging as an MRI contrast agent.

The term “detecting” or “detection” as used herein relates to a test aimed at assessing the presence or absence of a target enzyme in a sample.

The term “fluorophore” refers to an organic molecule with the ability to absorb light at a particular wavelength and then emit it at a higher wavelength. To achieve this, photons of light from an excitation source are absorbed by the fluorophore's electrons, raising their energy level and causing them to move to an excited state. When the electron relaxes back to the ground state, energy is released in the form of photons. Fluorophores can be broadly categorized as organic dyes (e.g., fluorescein, rhodamine, AMCA), biological fluorophores (e.g., green fluorescent protein, phycoerythrin, allophycocyanin) and quantum dots. For example, in certain embodiments, the fluorophore used for imaging senescent cells is (E)-2-(2-(6-hydroxy-2,3-dihydro-1H-xanthen-4-yl)vinyl)-3,3-dimethyl-1-propyl-3H-indol-1-ium or “HXPI”.

The term “probe” refers to a means for detecting an analyte or enzyme. In the context of the disclosure, probes can e.g. be used to detect senescence-associated #-galactosidase (SA-β-Gal). For the purpose of detectability, probes typically carry labels such as a fluorophore, radioactive isotope or paramagnetic metal ion.

The term “radiotracer” refers to a chemical compound that carries at least one radionuclide so by detecting its radioactive decay, the molecule can be tracked in human or living animals using imaging technologies such as PET or SPECT. The term “radionuclide” or “radioisotope” refers to a nuclide that has excess nuclear energy, which is spontaneously released in the form of radiation.

The terms “senescent” or “senescence” refers to a process that halts cell proliferation, acts as an endogenous tumor suppression mechanism and is a cellular response to various stresses, including DNA damage, chromatin perturbation, and activation of oncogenes. Increasing evidence has revealed that senescence is implicated in aging and age-related diseases. “Senescence markers” refers to the biomarkers for senescence including overexpression of cell cycle inhibitors, such as p16, p21, and p53, as well as senescence-associated β-Galactosidase (SA-β-Gal), which is derived from the increased lysosomal content of senescent cells.

The term “self-immolative linker” refers to moieties which disassemble or degrade, typically spontaneously or in response to an activation event. An example of a self-immolative linker is 4-(hydroxymethyl)phenol. Other examples of suitable self-immolative linkers include, but are not limited to, the structure containing substituents on the phenyl ring of 4-(hydroxymethyl)phenol. Other known self-immolative linkers known in the art may be implemented into the probes according to the teachings herein, including those taught in Chen, X. Z.; Ma, X. D.; Zhang, Y. Y.; Gao, G.; Liu, J. J.; Zhang, X. Y.; Wang, M. A.; Hou, S. C., Ratiometric fluorescent probes with a self-immolative spacer for real-time detection of beta-galactosidase and imaging in living cells. Anal Chim Acta 2018, 1033, 193-198; and Kwan, D. H.; Chen, H. M.; Ratananikom, K.; Hancock, S. M.; Watanabe, Y.; Kongsaeree, P. T.; Samuels, A. L.; Withers, S. G., Self-immobilizing fluorogenic imaging agents of enzyme activity. Angewandte Chemie 2011, 50 (1), 300-3.

### DETAILED DESCRIPTION

The hallmarks of senescence include morphological changes such as enlarged and flattened cell shape and compromised nuclear membrane; overexpression of cell cycle inhibitors such as p16, p21 and p53²⁰⁻²⁴; chromatin and nuclear alterations²⁵⁻³¹; metabolic changes such as dysfunctional mitochondria³²⁻³⁴, increased lysosomal compartment characterized by overexpression of senescence-associated β-galactosidase (SA-β-Gal)³⁵⁻³⁷ and senescence-associated secretory phenotype (SASP)³⁸⁻⁴¹. These markers are usually used in combinations to evaluate senescence in vitro. However, there are limited methods for evaluating senescence in vivo due to the complex biological environment and limited sensitivity of the current available tools.

Presented herein is a class of imaging probes for SA-β-Gal based on quinone methide chemistry for different in vivo imaging modalities including near-infrared (NIR), positron emission tomography (PET), single photon emission computed tomography (SPECT), and magnetic resonance (MR) imaging. These probes have a common β-galactoside moiety that can be activated by SA-β-Gal followed by the formation of a quinone methide intermediate that can be captured by nucleophilic residues such as amines or thiols on intracellular proteins via covalent bonds. This mechanism improves the retention of the activated probe at the site of activation while the un-activated probes diffuse away, providing high signal to background ratio and pro longed imaging time window.

In a specific embodiment, disclosed is an NIR probe for detecting senescent cells. The probe comprises or consists of a target specific moiety, a fluorophore, a self-immolative linker, and a self-immobilizing moiety. In specific examples, the self-immobilizing moiety is monofluoromethyl, carbamate or carbonate.

### Overview

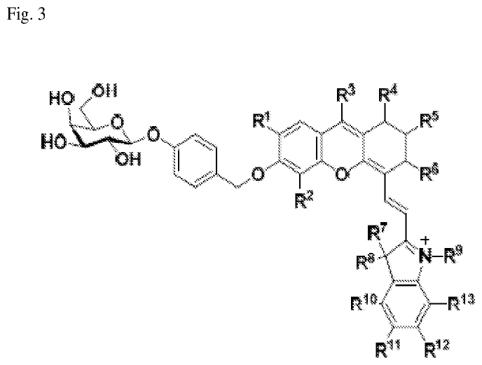

The Cui group has previously reported a self-immobilizing NIR probe (NIR-BG2) for SA-β-Gal⁴²⁻⁴³. The HXPI fluorophore on this probe was incorporated with a difluoromethyl handle which assists the formation of the quinone methide intermediate upon probe activation. The NIR-BG2 probe was tested in drug-induced senescence cell and animal models. To further improve the labeling efficiency of this NIR probe, new alterations have been developed including implementation of alternative functional groups such as monofluoromethyl, methylene carbamate or carbonate on HXPI. These new probes possess better labeling efficiency and stability compared to previous generation. In addition, certain embodiments of the new probes disclosed herein have dual self-immobilizing handles (R¹ and R², FIG. 3) to improve the probability of the activated probe being captured by intracellular proteins which can result in even higher contrast and sensitivity.

In certain examples, two carbamate analogues were synthesized: NIR-BG3 carrying ethyl carbamate and NIR-BG4 carrying an endocyclic carbamate. To compare these two analogues with NIR-BG2, the three probes were incubated with recombinant β-galactosidase (p-gal) and bovine serum albumin (BSA) and the labeling efficiency of these probes on P-gal and BSA was compared using gel electrophoresis.

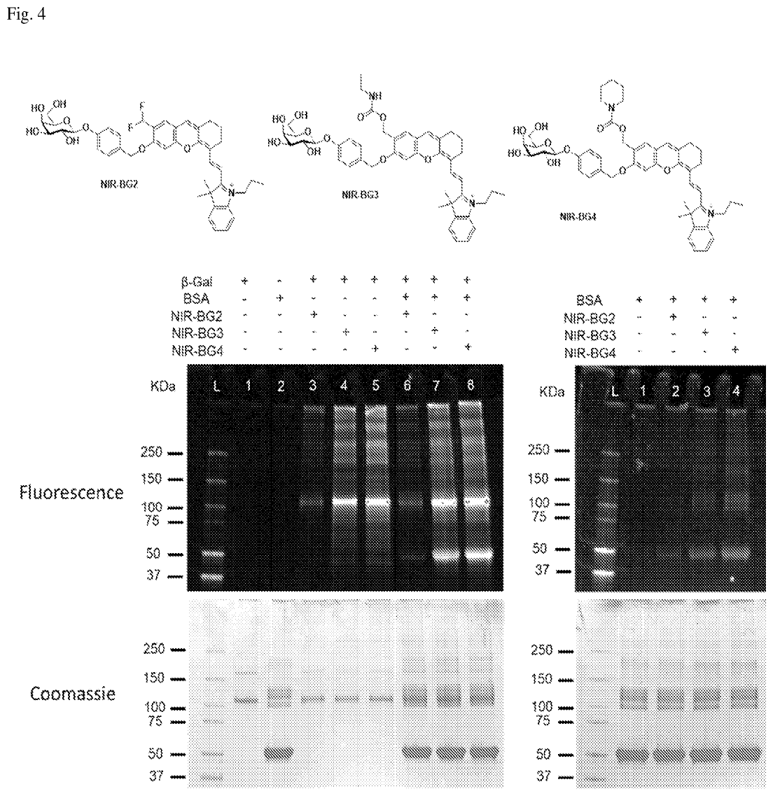

As shown in FIG. 4, NIR-BG3 and NIR-BG4 both show significantly higher labeling efficiency while showing slightly higher nonspecific labeling of BSA. NIR-BG2 and NIR-BG3 were also compared in CT26.WT and CT26.CL25 that are stably transfected with LacZ. Both NIR-BG2 and NIR-BG3 showed strong contrast for CT26.CL25 cells over CT26.WT, suggesting both probes can be specifically activated by β-gal and retained in CT26.CL25. Compared with NIR-BG2, NIR-BG3 showed overall higher fluorescence and higher background at the same time as shown by flow cytometry. These results demonstrate that the new carbamate analogues disclosed herein have higher labeling efficiency, but future optimization may be needed to minimize background.

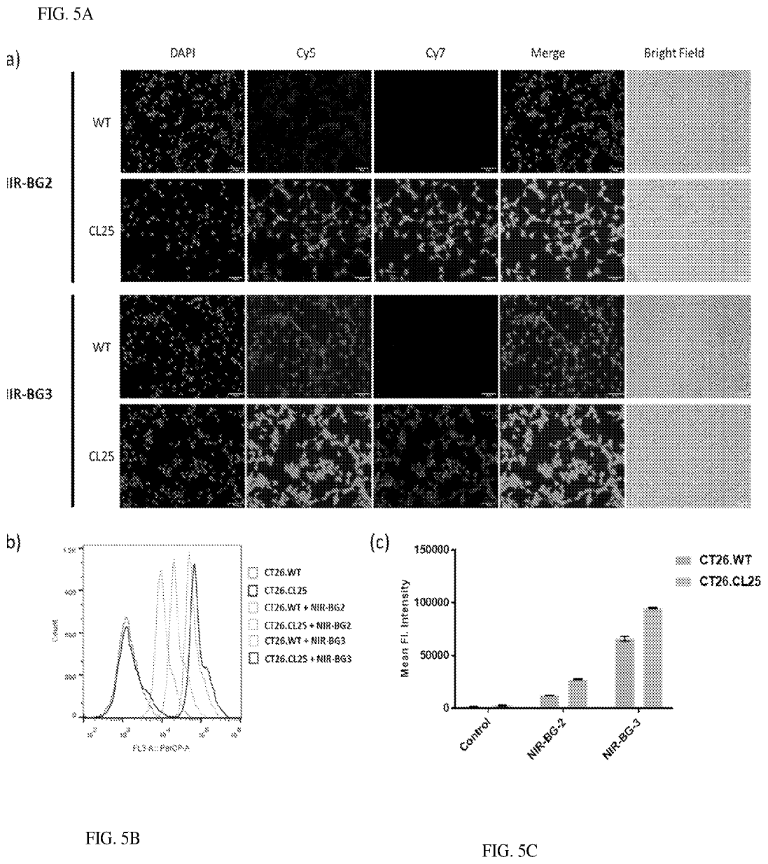

FIG. 5 shows that NIR-BG3 fluorescent intensity is much higher than NIR-BG2 (see FIG. 5A and FIG. 5C). The mean intensity is about 2-fold higher for NIR-BG3 compared to NIR-BG2.

In addition to the NIR probes, disclosed herein are PET and MRI probes for in vivo detection of SA-β-gal. Compared to fluorescence imaging, PET and MRI are more clinically relevant and have different advantages over fluorescence imaging. PET is highly sensitive and only require a very small amount of imaging probe and MRI has high spatial resolution.

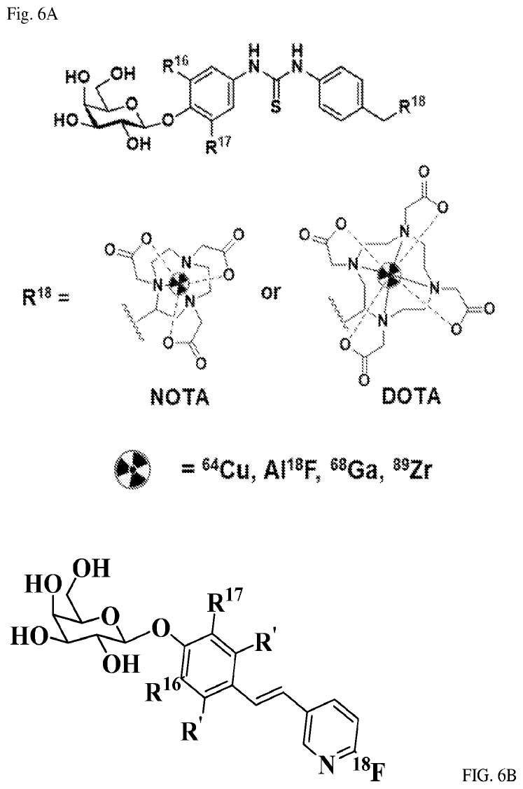

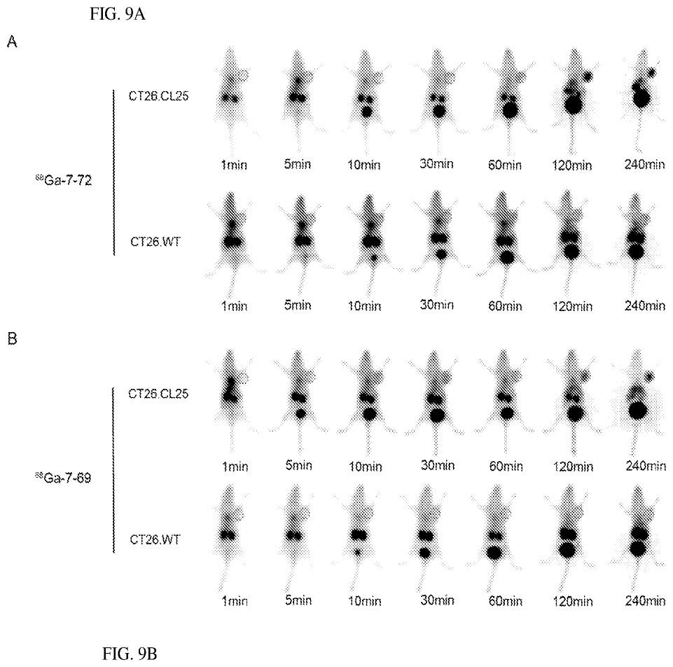

FIG. 9 shows the results of PET imaging of subcutaneous tumors of CT26.CL25 or CT26 wild type that were inoculated on the right shoulder of the mice. Imaging probes (100 uCi) were injected via tail vein. 1-Hour dynamic PET imaging was performed followed by twice static PET/CT scan at 2 h and 4 h post injection. PET SA-β-Gal probe as shown in FIG. 6A, was used, wherein R18=NOTA, and ⁶⁸Ga is the radioactive metal chelated to NOTA.

### Chemical Synthesis of NIR-BG3 and NIR-BG4

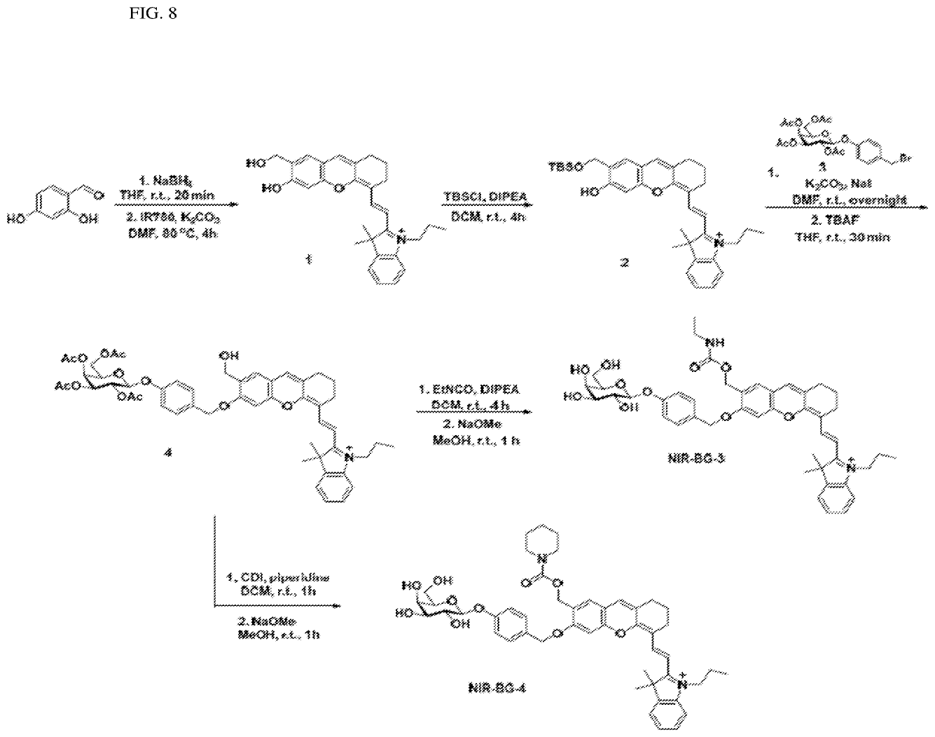

FIG. 8 shows the synthesis scheme for NIR-BG3 and NIR-BG4, which is further detailed below.

General Information: All chemicals were purchased from commercial sources unless otherwise noted. HRMS samples were analyzed on Waters LCT Premier Mass Spectrometer. High-performance liquid chromatography (HPLC) was performed on a Dionex Ultimate 300 HPLC System (Thermo Scientific) equipped with a GP50 gradient pump and an in-line diode array UV-Vis detector. Reverse-phase C18 columns were used with acetonitrile/water gradient mobile phase containing 0.1% trifluoroacetic acid. NMR spectra were recorded on Bruker instruments (500 MHz and 600 MHz for ¹H NMR, 126 MHz and 151 MHz for ¹³C NMR) and internally referenced to the residual solvent signals (¹H: 67.26; ¹³C: δ 77.16 for CDCl3, ¹H: δ 3.31; ¹³C: δ 49.0 for CD3OD respectively). NMR chemical shifts (δ) and the coupling constants (J) for ¹H and ¹³C NMR are reported in parts per million (ppm) and in Hertz, respectively. The following conventions are used for multiplicities: s, singlet; d, doublet; t, triplet; m, multiplet; and dd, doublet of doublet.

Compound 1: 2,4-dihydrobenzaldehyde (600 mg, 4.35 mmol) was dissolved in THF and was added NaBH₄ (198 mg, 5.22 mmol). The reaction was stirred at room temperature for 1 h and was quenched with MeOH. The solvent was removed in vacuo and the crude intermediate was used in the next step without further purification. IR-780 iodide (1.45 g, 2.2 mmol) and K₂CO₃ (304 mg, 2.2 mmol) were added to the crude intermediate in anhydrous DMF, and the reaction was stirred at 80° C. for 4 h. The reaction mixture was extracted with ethyl acetate and water. The organic layer was dried over Na₂SO₄ and was concentrated in vacuo. The crude product was purified through flash column chromatography (SiO₂; 0%-10% MeOH/DCM) to afford the titled compound. Yield: 320 mg, 33%.

¹H NMR (600 MHz, DMSO-d6) δ 11.10 (s, 1H), 8.57 (d, J 14.7 Hz, 1H), 7.74 (d, J=7.5, 1H), 7.68-7.62 (m, 2H), 7.58 (s, 1H), 7.52 (t, J=7.7 Hz, 1H), 7.42 (t, J=7.5 Hz, 1H), 6.97 (s, 1H), 6.49 (d, J 14.7 Hz, 1H), 4.53 (s, 2H), 4.34 (t, J=7.3 Hz, 2H), 2.75 (t, J=6.1 Hz, 2H), 2.68 (t, J=6.2 Hz, 2H), 1.83 (m, 4H), 1.75 (s, 6H), 0.99 (t, J=7.4 Hz, 3H).

¹³C NMR (151 MHz, DMSO) δ 176.76, 162.00, 159.28, 153.51, 144.64, 142.19, 142.05, 135.95, 129.44, 129.30, 126.88, 126.51, 126.25, 123.18, 114.68, 114.25, 113.24, 103.35, 101.49, 58.06, 50.44, 46.21, 28.77, 28.10, 24.07, 21.22, 20.49, 11.52.

LCMS (ESI): calc'd for C₂₉H₃₂NO₃⁺ [M⁺] 442.2377; found: 442.27.

Compound 2: Compound 1 (320 mg, 0.72 mmol) in DMF and was added DIPEA (450 μL) and TBSCl (330 mg, 2.16 mmol). The reaction was stirred at room temperature for 2 h and was extracted with ethyl acetate and water. The organic layer was dried over Na₂SO₄ and was concentrated in vacuo. The crude product was purified through flash column chromatography (SiO₂; 0%-5% MeOH/DCM) to afford the titled compound. Yield: 300 mg, 75%.

¹H NMR (600 MHz, DMSO-d6) δ 11.08 (s, 1H), 8.57 (d, J=14.7 Hz, 1H), 7.76 (d, J=7.5 Hz, 1H), 7.69-7.62 (m, 2H), 7.55-7.49 (m, 2H), 7.47-7.39 (m, 1H), 6.95 (s, 1H), 6.50 (d, J=14.8 Hz, 1H), 4.71 (s, 2H), 4.34 (t, J=7.3 Hz, 2H), 2.75 (t, J=6.0 Hz, 2H), 2.69 (t, J=6.2 Hz, 2H), 1.88-1.78 (m, 4H), 1.75 (s, 6H), 1.00 (t, J=7.4 Hz, 3H), 0.94 (s, 9H), 0.12 (s, 6H).

¹³C NMR (151 MHz, DMSO-d6) δ176.96, 161.82, 158.92, 153.59, 144.79, 142.16, 142.11, 135.68, 129.41, 129.31, 127.93, 126.98, 126.45, 126.39, 123.22, 114.72, 114.26, 113.33, 103.62, 59.82, 50.52, 46.28, 28.73, 28.06, 26.34, 24.07, 21.25, 20.48, 18.59, 11.52, −4.85.

LCMS (ESI): calc'd for C₃₅H₄₆NO₃Si⁺ [M⁺]556.3241.2377; found: 556.37.

Compound 3 was prepared according to literature. (O. Redy-Keisar, E. Kisin-Finfer, S. Ferber, R. Satchi-Fainaro, D. Shabat, Synthesis and use of QCy7-derived modular probes for the detection and imaging of biologically relevant analytes, Nat Protoc, 9(2014) 27-36.)

Compound 4: Compound 2 (30 mg, 54 μmol), Compound 3 (83.6 mg, 162 μmol), K₂CO₃ (22 mg, 162 mmol) and NaI (24 mg, 162 mmol) were stirred in anhydrous DMF at room temperature overnight. The reaction was extracted with ethyl acetate and water and the organic layer was dried over Na₂SO₄ and was concentrated in vacuo. The crude product was used in the next step without further purification. The crude product from the coupling reaction was dissolved in THF and was added TBAF (1M in THF solution, 40 μL). The reaction was stirred at room temperature for 30 min before the solvent was removed in vacuo. The crude product was purified through flash column chromatography (SiO₂; 0%-5% MeOH/DCM). Yield: 20 mg, 42% over 2 steps.

¹H NMR (600 MHz, DMSO-d6) δ 8.59 (d, J=14.8 Hz, 1H), 7.78 (d, J=7.5 Hz, 1H), 7.68 (d, J=8.0 Hz, 1H), 7.63 (d, J=6.8 Hz, 2H), 7.57-7.50 (m, 3H), 7.45 (t, J=7.5 Hz, 1H), 7.20 (s, 1H), 7.07 (d, J=7.5 Hz, 2H), 6.55 (d, J=14.8 Hz, 1H), 5.50 (d, J=7.8 Hz, 1H), 5.35 (dd, J=3.4, 1.1 Hz, 1H), 5.232-5.26 (m, 3H), 5.22 (dd, J=10.4, 7.8 Hz, 1H), 4.56 (s, 2H), 4.46-4.41 (m, 1H), 4.38 (t, J=7.3 Hz, 2H), 4.16-4.05 (m, 2H), 2.75 (t, J=6.0 Hz, 2H), 2.69 (t, J=6.1 Hz, 2H), 2.14 (s, 3H), 2.02 (s, 3H), 1.99 (s, 3H), 1.95 (s, 3H), 1.85 (m, 4H), 1.80 (d, J=4.1 Hz, 6H), 1.00 (t, J=7.3 Hz, 3H).

¹³C NMR (151 MHz, DMSO-d6) δ177.49, 170.42, 170.26, 170.03, 169.68, 161.53, 158.90, 158.81, 158.67, 158.44, 158.22, 156.80, 153.48, 144.90, 142.37, 142.08, 134.73, 131.15, 130.32, 129.99, 129.33, 127.49, 127.28, 125.89, 123.22, 117.63, 116.98, 115.68, 115.45, 114.39, 113.53, 104.25, 99.91, 98.09, 70.80, 70.60, 70.23, 68.81, 67.65, 61.70, 58.02, 50.69, 46.36, 28.90, 28.16, 24.16, 21.32, 20.91, 20.89, 20.85, 20.81, 20.39, 11.50.

LCMS (ESI): calc'd for C₅₀H₅₆NO⁺ [M⁺]878.3746; found: 878.48.

NIR-BG3: Compound 4 (10 mg, 11.4 μmol) in DCM was added DIPEA (20 μL) and ethyl isocyanate (9.1 μL, 114 μmol). The reaction was stirred at room temperature for 4 h and was quenched with MeOH. The solvent was removed in vacuo to afford crude intermediate, which was used in the next step without further purification. The crude compound in MeOH was added catalytic amount of NaOMe. The reaction was stirred at room temperature for 1 h and the product was purified through HPLC. Yield: 1.2 mg, 18% over 2 steps.

¹H NMR (600 MHz, DMSO-d6) δ 8.58 (d, J=14.8 Hz, 1H), 7.81 (d, J=7.5 Hz, 1H), 7.71 (d, J=8.1 Hz, 1H), 7.55 (t, J=7.8, 1H), 7.53 (s, 1H), 7.52 (s, 1H), 7.49-7.46 (m, 3H), 7.28 (t, J=5.6 Hz, 1H), 7.22 (s, 1H), 7.09 (d, J=8.7 Hz, 2H), 6.59 (d, J=14.9 Hz, 1H), 5.31 (s, 2H), 5.05 (s, 2H), 4.83 (d, J=7.7 Hz, 1H), 4.40 (t, J=7.2 Hz, 2H), 3.70 (d, J=3.3 Hz, 1H), 3.60-3.45 (m, 4H), 3.40 (dd, J=9.5, 3.3 Hz, 1H), 3.07-2.99 (m, 2H), 2.73 (t, J=6.0 Hz, 2H), 2.69 (t, J=6.1 Hz, 2H), 1.88-1.83 (m, 4H), 1.81 (s, 3H), 1.80 (s, 3H), 1.03 (t, J=7.2 Hz, 3H), 1.00 (t, J=7.4 Hz, 3H).

¹³C NMR (151 MHz, DMSO) δ 178.00, 160.94, 159.54, 158.41, 158.19, 157.99, 156.27, 154.10, 145.15, 142.54, 142.00, 133.55, 129.74, 129.68, 129.36, 128.31, 127.87, 127.57, 124.58, 123.32, 116.78, 115.31, 114.46, 113.76, 104.99, 101.52, 100.59, 76.01, 73.76, 70.70, 70.61, 68.56, 60.83, 60.54, 50.89, 46.52, 35.59, 30.08, 28.90, 28.10, 24.13, 21.41, 20.34, 15.53, 11.51.

LCMS (ESI): calc'd for C₄₅H₅₃N₂O⁺ [M⁺] 781.3695; found: 781.42.

NIR-BG4: Compound 4 (10 mg, 11.4 μmol) in DCM was added CDI (2.8 mg, 17.1 μmol) and the reaction was stirred at room temperature for 1 h. The solvent was removed by rotary evaporation and the crude compound was dissolved in MeOH and was added catalytic amount of NaOMe. The reaction was stirred at room temperature for 1 h and the product was purified through HPLC. Yield: 8 mg, 85% over 2 steps.

¹H NMR (600 MHz, DMSO-d6) δ 8.59 (d, J 14.9 Hz, 111), 7.81 (d, J=7.5 Hz, 1H), 7.71 (d, J=8.1 Hz, 1H), 7.58-7.52 (m, 3H), 7.50-7.45 (m, 3H), 7.23 (s, 1H), 7.09 (d, J=8.7 Hz, 2H), 6.59 (d, J 14.9 Hz, 1H), 5.32 (s, 2H), 5.09 (s, 2H), 4.83 (d, J=7.7 Hz, 1H), 4.40 (t, J=7.3 Hz, 2H), 3.70 (d, J=3.3 Hz, 1H), 3.60-3.52 (m, 3H), 3.48 (dt, J=9.7, 5.0 Hz, 1H), 3.40 (dd, J=9.5, 3.3 Hz, 1H), 3.38-3.31 (m, 4H), 2.73 (t, J=6.0 Hz, 2H), 2.69 (t, J=6.2 Hz, 2H), 1.89-1.82 (m, 4H), 1.80 (d, J=2.4 Hz, 6H), 1.55 (t, J=7.6, 4.6 Hz, 2H), 1.48-1.42 (m, 4H), 1.00 (t, J=7.4 Hz, 3H).

¹³C NMR (151 MHz, DMSO) δ 177.98, 161.00, 159.54, 158.52, 158.30, 158.08, 158.01, 154.71, 154.15, 145.17, 142.54, 142.00, 133.69, 129.73, 129.69, 129.36, 128.19, 127.82, 127.56, 124.27, 123.32, 116.77, 115.34, 114.46, 113.75, 104.94, 101.53, 100.62, 76.01, 73.77, 70.71, 70.58, 68.56, 61.77, 60.83, 56.31, 50.88, 46.52, 44.83, 32.58, 30.07, 28.89, 28.10, 28.09, 24.24, 24.13, 21.40, 20.36, 11.51.

LCMS (ESI): calc'd for C₄₈H₅₇N₂O⁺ [M⁺] 821.4008; found: 821.46.

### Imaging Modalities and Related Probes

Disclosed are imaging probes for detecting cellular senescence both in vitro and in vivo and the methods of use thereof in conjunction with imaging modalities. Methods and devices that may be used as imaging modalities include, but are not limited to, near infrared fluorescence imaging, whole body scan such as position emission tomography (PET), single photon emission computed tomography (SPECT) and magnetic resonance imaging (MRI).

In certain embodiments, the imaging modality for detecting the probes in vivo is NIR fluorescence. NIR fluorescent probes offer high penetration depth, minimal photodamage to tissues, decreased background autofluorescence, and have been applied in noninvasive detection and imaging of biological targets in vivo. NIR fluorophores are fluorophores with excitation and emission spectra in the near infrared range. Examples of suitable NIR fluorophore reporters include, but are not limited to, hemicyanine derivatives. Hemicyanine derivatives, for example, have excellent stability, low toxicity, and fluorescence emission in NIR range upon activation. The fluorophore has demonstrated its suitability for fluorescence imaging of various biomolecules, such as the detection of P-Lactamase in Staphylococcus aureus, the detection of nitroxyl (HNO) and palladium in live cells, the detection of nitroreductase, y-glutamyl transpeptidase and tyrosinase in zebrafish. More recently, the hemicyanine NIR dyes have been used for in vivo detection of cysteine, alkaline phosphatase in tumor models, superoxide radical anion, hydrogen sulfide, hydrogen polysulfides and y-glutamyl transpeptidase in mice models. A specific example of an NIR fluorophore is (E)-2-(2-(6-hydroxy-2,3-dihydro-1H-xanthen-4-yl)vinyl)-3,3-dimethyl-1-propyl-3H-5 indol-1-ium (HXPI).

In certain embodiments, the probes are NIR probes for detecting senescent cells in a subject as shown in FIG. 3. The NIR probes are comprised of a target specific moiety, a fluorophore, a self-immolative linker, and a self-immobilizing moiety. The self-immobilizing moiety is selected from the group consisting of monofluoromethyl, carbamate and carbonate. FIG. 3 shows the general structure of the NIR probe embodiments. R¹ and R² are independently selected from: H, CH₂OCON(CH₂)a, CH₂OCOOR¹⁵, C1-C10 alkyl, C5-C20 aryl, C₁-C₁₀ alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO⁻, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H. R³-R¹⁵ are independently selected from: H, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO⁻, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H. The variable a varies from 2 to 20. The variables b and c independently vary from 1 to 20. Bm is selected from the group consisting of bioactive peptides containing 2 to 30 amino acid units, proteins, antibody fragments, mono- and oligosaccharides, and bioactive drug compounds.

In a specific embodiment, the NIR probes have a chemical structure of:

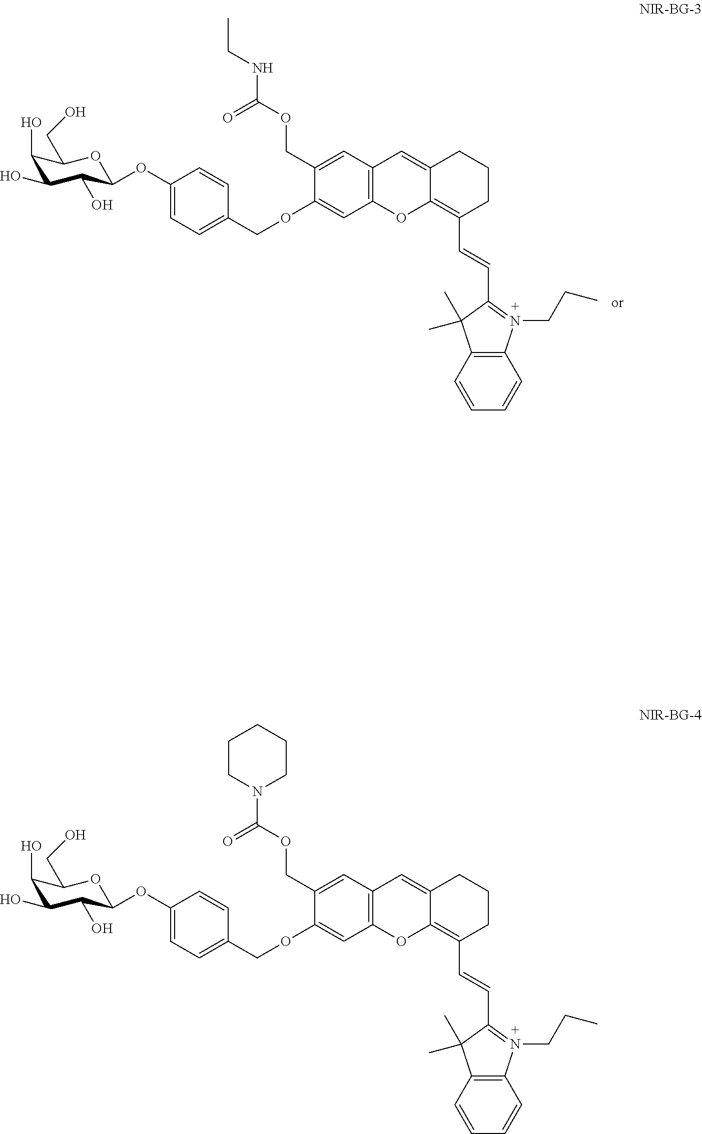

In certain embodiments, the imaging probes are PET probes for in vivo imaging of SA-β-Gal. Positron emission tomography (PET) is a functional imaging technique that uses radioactive substances known as radiotracers to visualize and measure changes in metabolic processes, and in other physiological activities including blood flow, regional chemical composition, and absorption.

Different tracers are used for various imaging purposes, depending on the target process within the body. Examples of suitable radiotracers include, but are not limited to, a chelator such as 1,4,7-triazacyclononane-1,4,7-triacetic acid (NOTA) or (1,4,7,10-tetraazacyclododecane-1,4,7,10-tetraacetic acid) DOTA chelated to a radioisotope, such as, but not limited to ⁶⁴Cu, Al¹⁸F, ⁶⁸Ga, and ⁸⁹Zr.

In certain embodiments, the PET probe for detecting senescent cells comprises a target specific moiety, a radiotracer, and a self-immobilizing moiety. In some embodiments, the target specific moiety comprises a senescence marker. In a specific embodiment, the senescence marker is senescence-associated β-galactosidase. FIGS. 6A and 6B show the general structures of PET probe embodiments. In FIGS. 6A and 6B, R¹⁶ and R¹⁷ are independently selected from: H, CH₂F, CHF₂, CH₂OCONHR¹⁹ CH₂OCON(CH₂)a, CH₂OCOOR²⁰, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO₃⁻, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H. R¹⁹-R²⁰ are independently selected from: H, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂—(CH₂OCH₂)b-CH₂—OH, —(CH₂)c-CO₂H, —(CH₂)c-SO₃⁻, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H. The variable a varies from 2 to 20. The variables b and c independently vary from 1 to 20. Bm is selected from the group consisting of bioactive peptides containing 2 to 30 amino acid units, proteins, antibody fragments, mono- and oligosaccharides and bioactive drug compounds. In FIG. 6A, R¹⁸ is NOTA or DOTA. The radionucleotide is selected from ⁶⁴Cu, Al, ¹⁸F, ⁶⁸Ga, and ⁸⁹Zr.

In certain embodiments, the imaging probes are MRI probes for in vivo imaging of SA-β-Gal. In certain embodiments, the MRI probe for detecting senescent cells comprises a target specific moiety, a contrast agent, and a self-immobilizing moiety. The target specific moiety comprises a senescence marker, and in a specific embodiment, the senescence marker is senescence-associated β-galactosidase. The clinical utility of contrast agents is well established. The most commonly used MRI probes are based on chelates of gadolinium. In a specific embodiment, the MRI probe disclosed contains a gadolinium complex.

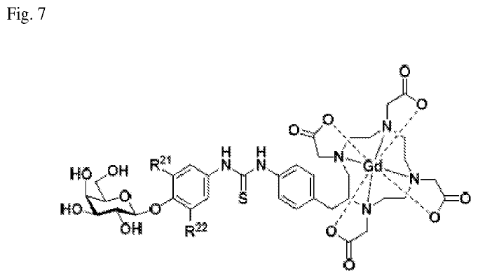

FIG. 7 shows the general structure of other MRI probe embodiments. In FIG. 7, R²¹ and R²² are independently selected from: H, CH₂F, CHF₂, CH₂OCONHR²³ CH₂OCON(CH₂)a, CH₂OCOOR²⁴, C1-C10 alkyl, C5-C20 aryl, C1-C10 alkoxyl, C1-C10 polyalkoxyalkyl, C1-C20 polyhydroxyalkyl, C5-C20 polyhydroxyaryl, C₁-C₁₀ aminoalkyl, cyano, nitro, halogens, saccharides, peptides, —CH₂(CH₂OCH₂)b-CH₂—OH, —(CH₂)c-CO₂H, —(CH₂)c-SO₃, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂ (CH₂OCH₂)b-CO₂H. R²³-R²⁴ are independently selected from: H, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO₃, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H. The variable a varies from 2 to 20. The variables b and c independently vary from 1 to 20. Bm is selected from the group consisting of bioactive peptides containing 2 to 30 amino acid units, proteins, antibody fragments, mono- and oligosaccharides and bioactive drug compounds.

### REFERENCES

1. Campisi, J.; d'Adda di Fagagna, F., Cellular senescence: when bad things happen to good cells. Nat. Rev. Mol. Cell Biol. 2007, 8 (9), 729-40.
2. Munoz-Espin, D.; Serrano, M., Cellular senescence: from physiology to pathology. Nat. Rev. Mol. Cell Biol. 2014, 15 (7), 482-96.
3. Childs, B. G.; Durik, M.; Baker, D. J.; van Deursen, J. M., Cellular senescence in aging and age-related disease: from mechanisms to therapy. Nat Med 2015, 21 (12), 1424-35.
4. van Deursen, J. M., The role of senescent cells in ageing. Nature 2014, 509 (7501), 439-46.
5. Childs, B. G.; Baker, D. J.; Wijshake, T.; Conover, C. A.; Campisi, J.; van Deursen, J. M., Senescent intimal foam cells are deleterious at all stages of atherosclerosis. Science 2016, 354 (6311), 472-477.
6. Schafer, M. J.; White, T. A.; Iijima, K.; Haak, A. J.; Ligresti, G.; Atkinson, E. J.; Oberg, A. L.; Birch, J.; Salmonowicz, H.; Zhu, Y.; Mazula, D. L.; Brooks, R. W.; Fuhrmann-Stroissnigg, H.; Pirtskhalava, T.; Prakash, Y. S.; Tchkonia, T.; Robbins, P. D.; Aubry, M. C.; Passos, J. F.; Kirkland, J. L.; Tschumperlin, D. J.; Kita, H.; LeBrasseur, N. K., Cellular senescence mediates fibrotic pulmonary disease. Nat Commun 2017, 8, 14532.
7. Aguayo-Mazzucato, C.; Andle, J.; Lee, T. B., Jr.; Midha, A.; Talemal, L.; Chipashvili, V.; Hollister-Lock, J.; van Deursen, J.; Weir, G.; Bonner-Weir, S., Acceleration of beta Cell Aging Determines Diabetes and Senolysis Improves Disease Outcomes. Cell Metab 2019, 30 (1), 129-142 e4.
8. Thompson, P. J.; Shah, A.; Ntranos, V.; Van Gool, F.; Atkinson, M.; Bhushan, A., Targeted Elimination of Senescent Beta Cells Prevents Type 1 Diabetes. Cell Metab 2019, 29 (5), 1045-1060 e10.
9. Bussian, T. J.; Aziz, A.; Meyer, C. F.; Swenson, B. L.; van Deursen, J. M.; Baker, D. J., Clearance of senescent glial cells prevents tau-dependent pathology and cognitive decline. Nature 2018, 562 (7728), 578-582.
10. Zhang, P.; Kishimoto, Y.; Grammatikakis, I.; Gottimukkala, K.; Cutler, R. G.; Zhang, S.; Abdelmohsen, K.; Bohr, V. A.; Misra Sen, J.; Gorospe, M.; Mattson, M. P., Senolytic therapy alleviates Abeta-associated oligodendrocyte progenitor cell senescence and cognitive deficits in an Alzheimer's disease model. Nat Neurosci 2019, 22 (5), 719-728.
11. Jeon, 0. H.; Kim, C.; Laberge, R. M.; Demaria, M.; Rathod, S.; Vasserot, A. P.; Chung, J. W.; Kim, D. H.; Poon, Y.; David, N.; Baker, D. J.; van Deursen, J. M.; Campisi, J.; Elisseeff, J. H., Local clearance of senescent cells attenuates the development of post-traumatic osteoarthritis and creates a pro-regenerative environment. Nat Med 2017, 23 (6), 775-781.
12. Perez-Mancera, P. A.; Young, A. R.; Narita, M., Inside and out: the activities of senescence in cancer. Nat Rev Cancer 2014, 14 (8), 547-58.
13. Faget, D. V.; Ren, Q.; Stewart, S. A., Unmasking senescence: context-dependent effects of SASP in cancer. Nat Rev Cancer 2019, 19 (8), 439-453.
14. Lee, S.; Schmitt, C. A., The dynamic nature of senescence in cancer. Nat Cell Biol 2019, 21 (1), 94-101.
15. Munoz-Espin, D.; Canamero, M.; Maraver, A.; Gomez-Lopez, G.; Contreras, J.; Murillo-Cuesta, S.; Rodriguez-Baeza, A.; Varela-Nieto, I.; Ruberte, J.; Collado, M.; Serrano, M., Programmed cell senescence during mammalian embryonic development. Cell 2013, 155 (5), 1104-18.
16. Krizhanovsky, V.; Yon, M.; Dickins, R. A.; Hearn, S.; Simon, J.; Miething, C.; Yee, H.; Zender, L.; Lowe, S. W., Senescence of activated stellate cells limits liver fibrosis. Cell 2008, 134 (4), 657-67.
17. Jun, J. I.; Lau, L. F., The matricellular protein CCN1 induces fibroblast senescence and restricts fibrosis in cutaneous wound healing. Nat Cell Biol 2010, 12 (7), 676-85.
18. Xue, W.; Zender, L.; Miething, C.; Dickins, R. A.; Hernando, E.; Krizhanovsky, V.; Cordon-Cardo, C.; Lowe, S. W., Senescence and tumour clearance is triggered by p53 restoration in murine liver carcinomas. Nature 2007, 445 (7128), 656-60.
19. Kang, T. W.; Yevsa, T.; Woller, N.; Hoenicke, L.; Wuestefeld, T.; Dauch, D.; Hohmeyer, A.; Gereke, M.; Rudalska, R.; Potapova, A.; Iken, M.; Vucur, M.; Weiss, S.; Heikenwalder, M.; Khan, S.; Gil, J.; Bruder, D.; Manns, M.; Schirmacher, P.; Tacke, F.; Ott, M.; Luedde, T.; Longerich, T.; Kubicka, S.; Zender, L., Senescence surveillance of pre-malignant hepatocytes limits liver cancer development. Nature 2011, 479 (7374), 547-51.
20. Ben-Porath, I.; Weinberg, R. A., The signals and pathways activating cellular senescence. Int J Biochem Cell Biol 2005, 37 (5), 961-76.
21. Beausejour, C. M.; Krtolica, A.; Galimi, F.; Narita, M.; Lowe, S. W.; Yaswen, P.; Campisi, J., Reversal of human cellular senescence: roles of the p53 and p16 pathways. EMBO J 2003, 22 (16), 4212-22.
22. Chen, J., The Cell-Cycle Arrest and Apoptotic Functions of p53 in Tumor Initiation and Progression. Cold Spring Harb Perspect Med 2016, 6 (3), a026104.
23. Sharpless, N. E.; Sherr, C. J., Forging a signature of in vivo senescence. Nat Rev Cancer 2015, 15(7), 397-408.
24. Hernandez-Segura, A.; de Jong, T. V.; Melov, S.; Guryev, V.; Campisi, J.; Demaria, M., Unmasking Transcriptional Heterogeneity in Senescent Cells. Curr Biol 2017, 27 (17), 2652-2660 e4.
25. Di Micco, R.; Sulli, G.; Dobreva, M.; Liontos, M.; Botrugno, O. A.; Gargiulo, G.; dal Zuffo, R.; Matti, V.; d'Ario, G.; Montani, E.; Mercurio, C.; Hahn, W. C.; Gorgoulis, V.; Minucci, S.; d'Adda di Fagagna, F., Interplay between oncogene-induced DNA damage response and heterochromatin in senescence and cancer. Nature Cell Biology 2011, 13 (3), 292-302.
26. Zhang, R.; Chen, W.; Adams, P. D., Molecular dissection of formation of senescence-associated heterochromatin foci. Mol Cell Biol 2007, 27 (6), 2343-58.
27. Rodier, F.; Munoz, D. P.; Teachenor, R.; Chu, V.; Le, O.; Bhaumik, D.; Coppé, J.-P.; Campeau, E.; Beauséjour, C. M.; Kim, S.-H.; Davalos, A. R.; Campisi, J., DNA-SCARS: distinct nuclear structures that sustain damage-induced senescence growth arrest and inflammatory cytokine secretion. Journal of Cell Science 2011, 124 (1), 68-81.
28. d'Adda di Fagagna, F., Living on a break: cellular senescence as a DNA-damage response. Nat Rev Cancer 2008, 8 (7), 512-22.
29. Fumagalli, M.; Rossiello, F.; Clerici, M.; Barozzi, S.; Cittaro, D.; Kaplunov, J. M.; Bucci, G.; Dobreva, M.; Matti, V.; Beausejour, C. M.; Herbig, U.; Longhese, M. P.; d'Adda di Fagagna, F., Telomeric DNA damage is irreparable and causes persistent DNA-damage-response activation. Nature Cell Biology 2012, 14 (4), 355-365.
30. Herbig, U.; Jobling, W. A.; Chen, B. P. C.; Chen, D. J.; Sedivy, J. M., Telomere Shortening Triggers Senescence of Human Cells through a Pathway Involving ATM, p53, and p21CIP1, but Not p16INK4a. Molecular Cell 2004, 14 (4), 501-513.
31. de Oliveira Mann, C. C.; Kranzusch, P. J., cGAS Conducts Micronuclei DNA Surveillance. Trends in Cell Biology 2017, 27(10), 697-698.
32. Correia-Melo, C.; Marques, F. D. M.; Anderson, R.; Hewitt, G.; Hewitt, R.; Cole, J.; Carroll, B. M.; Miwa, S.; Birch, J.; Merz, A.; Rushton, M. D.; Charles, M.; Jurk, D.; Tait, S. W. G.; Czapiewski, R.; Greaves, L.; Nelson, G.; Bohlooly-Y, M.; Rodriguez-Cuenca, S.; Vidal-Puig, A.; Mann, D.; Saretzki, G.; Quarato, G.; Green, D. R.; Adams, P. D.; Zglinicki, T.; Korolchuk, V. I.; Passos, J. F., Mitochondria are required for pro-ageing features of the senescent phenotype. The EMBO Journal 2016, 35 (7), 724-742.
33. Vasileiou, P.; Evangelou, K.; Vlasis, K.; Fildisis, G.; Panayiotidis, M.; Chronopoulos, E.; Passias, P.-G.; Kouloukoussa, M.; Gorgoulis, V.; Havaki, S., Mitochondrial Homeostasis and Cellular Senescence. Cells 2019, 8 (7).
34. De Lange, T.; Passos, J. F.; Saretzki, G.; Ahmed, S.; Nelson, G.; Richter, T.; Peters, H.; Wappler, I.; Birket, M. J.; Harold, G.; Schaeuble, K.: Birch-Machin, M. A.; Kirkwood, T. B. L.; von Zglinicki, T., Mitochondrial Dysfunction Accounts for the Stochastic Heterogeneity in Telomere-Dependent Senescence. PLoS Biology 2007, 5 (5).
35. Dimri, G. P.; Lee, X.; Basile, G.; Acosta, M.; Scott, G.; Roskelley, C.; Medrano, E. E.; Linskens, M.; Rubelj, I.; Pereira-Smith, O., A biomarker that identifies senescent human cells in culture and in aging skin in vivo. Proceedings of the National Academy of Sciences 1995, 92 (20), 9363-9367.
36. Kurz, D. J.; Decary, S.; Hong, Y.; Erusalimsky, J. D., Senescence-associated (beta)-galactosidase reflects an increase in lysosomal mass during replicative ageing of human endothelial cells. Journal of Cell Science 2000,113 (20), 3613-3622.
37. Debacq-Chainiaux, F.; Erusalimsky, J. D.; Campisi, J.; Toussaint, O., Protocols to detect senescence-associated beta-galactosidase (SA-βgal) activity, a biomarker of senescent cells in culture and in vivo. Nature Protocols 2009, 4 (12), 1798-1806.
38. Coppé, J.-P.; Desprez, P.-Y.; Krtolica, A.; Campisi, J., The Senescence-Associated Secretory Phenotype: The Dark Side of Tumor Suppression. Annual Review of Pathology: Mechanisms of Disease 2010, 5 (1), 99-118.
39. Jakhar, R.; Crasta, K., Exosomes as Emerging Pro-Tumorigenic Mediators of the Senescence-Associated Secretory Phenotype. International Journal of Molecular Sciences 2019, 20(10).
40. Childs, B. G.; Durik, M.; Baker, D. J.; van Deursen, J. M., Cellular senescence in aging and age-related disease: from mechanisms to therapy. Nature Medicine 2015, 21 (12), 1424-1435.
41. Serrano, M.; Basisty, N.; Kale, A.; Jeon, O. H.; Kuehnemann, C.; Payne, T.; Rao, C.; Holtz, A.; Shah, S.; Sharma, V.; Ferrucci, L.; Campisi, J.; Schilling, B., A proteomic atlas of senescence-associated secretomes for aging biomarker development. PLOS Biology 2020, 18 (1).
42. Wang, Y.; Liu, J.; Ma, X.; Cui, C.; Deenik, P. R.; Henderson, P. K. P.; Sigler, A. L.; Cui, L., Real-time imaging of senescence in tumors with DNA damage. Scientfic Reports 2019, 9 (1).
43. Liu, J.; Ma, X.; Cui, C.; Chen, Z.; Wang, Y.; Deenik, P. R.; Cui, L., Noninvasive NIR imaging of senescence via in situ labeling. J. Med. Chem. 2021, 64 (24).

What is claimed is:

1. An NIR probe for detecting senescent cells comprising a target specific moiety, a fluorophore, a self-immolative linker, and a self-immobilizing moiety, wherein the self-immobilizing moiety is selected from the group consisting of monofluoromethyl, carbamate and carbonate.

2. The probe of claim 1, wherein the target specific moiety comprises a senescence marker.

3. The probe of claim 2, wherein the senescence marker comprises senescence-associated β-galactosidase.

4. The probe of claim 1, wherein the fluorophore comprises a hemicyanine derivative.

5. The probe of claim 4, wherein the hemicyanine derivative is HXPI.

6. The probe of claim 1, wherein the immobilizing moiety is selected from the group consisting of H, CH₂OCON(CH₂)a, CH₂OCOOR′, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)_{b}—CH₂—OH, —(CH₂)_{c}—CO₂H, —(CH₂)_{c}—SO₃⁺, —(CH₂)_{c}—CONH—Bm, —CH₂—(CH₂OCH₂)_{b}—CH₂—CONH—Bm, —(CH₂)_{c}—NHCO—Bm, —CH₂—(CH₂OCH₂)_{b}—CH₂—NHCO—Bm, —(CH₂)_{c}—OH, and —CH₂—(CH₂OCH₂)_{b}—CO₂H;
wherein R′ is selected from the group consisting of H, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO3−, —(CH₂)c-CONH—Bm, —CH₂ (CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H, and
wherein variable a is 2 to 20; variables b and c are, independently, 1 to 20; and Bm is selected from the group consisting of bioactive peptides containing 2 to 30 amino acid units, proteins, antibody fragments, mono- and oligosaccharides, and bioactive drug compounds.

7. The probe of claim 1, having a chemical structure comprising: 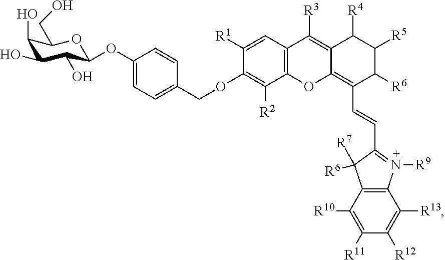
wherein R¹ and R² are independently selected from: H, CH₂OCON(CH₂)a, CH₂OCOOR₁₅, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO—, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H;
R3-R15 are independently selected from: H, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO−, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H;
variable a is 2 to 20;
variables b and c, are, independently, 1 to 20; and
Bm is selected from the group consisting of bioactive peptides containing 2 to 30 amino acid units, proteins, antibody fragments, mono- and oligosaccharides, and bioactive drug compounds.

8. The probe of claim 7 comprising one of the following structures:

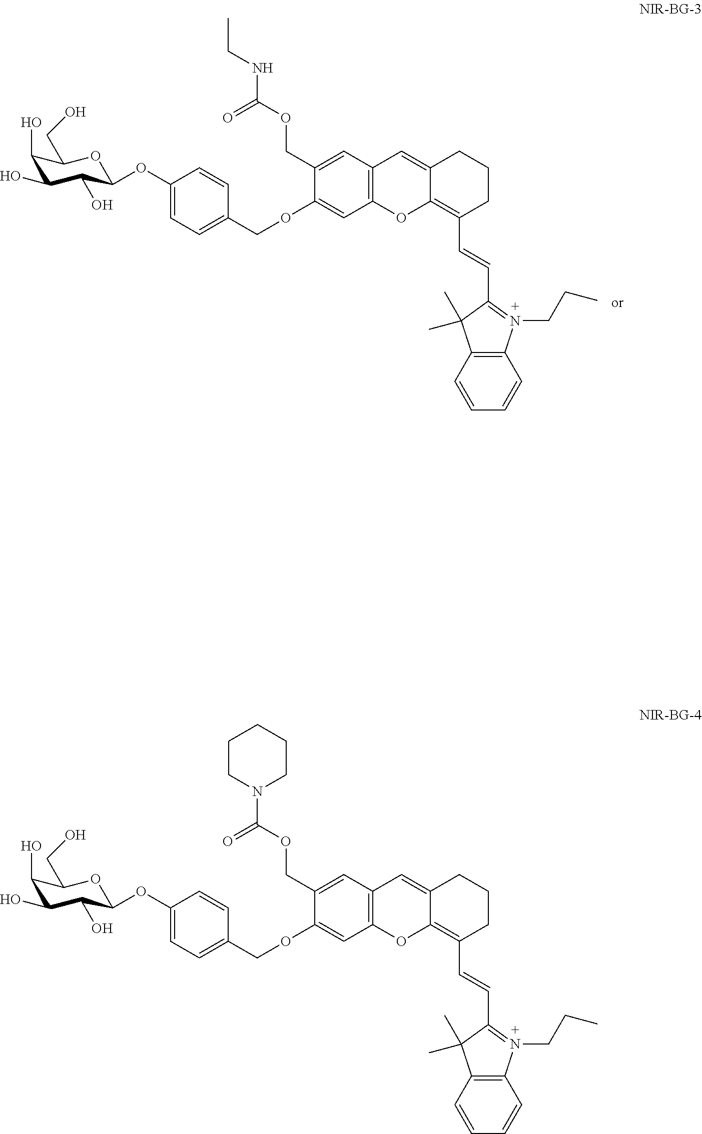

9. A PET probe for detecting senescent cells comprising a target specific moiety, a radiotracer, and a self-immobilizing moiety.

10. The probe of claim 9, wherein the target specific moiety comprises a senescence marker.

11. The probe of claim 10, wherein the senescence marker comprises senescence-associated p-galactosidase.

12. The probe of claim 9, wherein the self-immobilizing moiety is selected from the group consisting of H, CH₂F, CHF₂, CH₂OCONHR′, CH₂OCON(CH₂)a, CH₂OCOOR′, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —H₂(CH₂OCH₂)_{b}—CH₂—OH, —(CH₂)_{c}—CO₂H, —(CH₂)_{c}—SO₃−, —(CH₂)_{c}—CONH—Bm, —CH₂—(CH₂OCH₂)_{b}—CH₂—CONH—Bm, —(CH₂)_{c}—NHCO—Bm, —CH₂—(CH₂OCH₂)_{b}—CH₂—NHCO—Bm, —(CH₂)_{c}—OH and —CH₂—(CH₂OCH₂)_{b}—CO₂H;
wherein R′ is selected from the group consisting of H, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO3−, —(CH₂)c-CONH—Bm, —CH₂ (CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H, and
wherein variable a is 2 to 20; variables b and c, are, independently, 1 to 20; and Bm is selected from the group consisting of bioactive peptides containing 2 to 30 amino acid units, proteins, antibody fragments, mono- and oligosaccharides, and bioactive drug compounds.

13. The probe of claim 9, wherein the radiotracer comprises NOTA or DOTA.

14. The probe of claim 13, wherein NOTA and DOTA contain a radionuclide.

15. The probe of claim 14, wherein the radionuclide is selected from the group consisting of ⁶⁴Cu, Al¹⁸F, ⁶⁸Ga, and ⁸⁹ZR.

16. The probe of claim 9 having a chemical structure comprising: 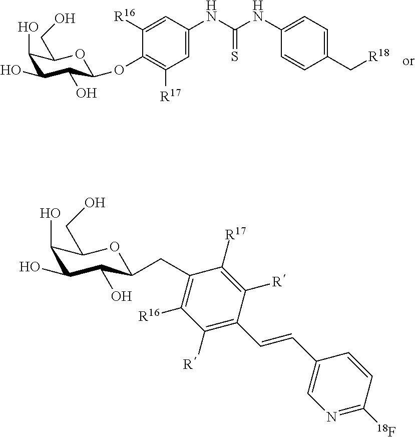
wherein R¹⁶ and R¹⁷ are independently selected from the group consisting of H, CH₂F, CHF₂, CH₂OCONHR′ CH₂OCON(CH₂)ₐ, CH₂OCOOR′, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —H₂(CH₂OCH₂)_{b}—CH₂—OH, —(CH₂)_{c}—CO₂H, —(CH₂)_{c}—SO₃₋, —(CH₂)_{c}—CONH—Bm, —CH₂—(CH₂OCH₂)_{b}—CH₂—CONH—Bm, —(CH₂)_{c}—NHCO—Bm, —CH₂—(CH₂OCH₂)_{b}—CH₂—NHCO—Bm, —(CH₂)_{c}—OH, and —CH₂—(CH₂OCH₂)_{b} CO₂H;
wherein R′ is selected from the group consisting of H, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO3−, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H, and
wherein variable a is 2 to 20; variables b and c, are, independently, 1 to 20; and Bm is selected from the group consisting of bioactive peptides containing 2 to 30 amino acid units, proteins, antibody fragments, mono- and oligosaccharides, and bioactive drug compounds.

17. The probe of claim 16, wherein R¹⁸ comprises NOTA or DOTA.

18. The probe of claim 17, wherein NOTA and DOTA comprise a radionuclide.

19. The probe of claim 18, wherein the radionuclide is selected from the group consisting of ⁶⁴Cu, Al¹⁸PF, ⁶⁸Ga, ⁸⁹Zr.

20. An MRI probe for detecting senescent cells comprising a target specific moiety, a contrast agent, and a self-immobilizing moiety.

21. The probe of claim 20, wherein the target specific moiety comprises a senescence marker.

22. The probe of claim 20 or 21, wherein the senescence marker comprises senescence-associated β-galactosidase.

23. The probe of any of claims 20-22, wherein the contrast agent contains Gadolinium.

24. The probe of any of claims 20-23, wherein the self-immobilizing moiety is selected from the group consisting of H, CH₂F, CHF₂, CH₂OCONHR′, CH₂OCON(CH₂)a, CH₂OCOOR′, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)_{b}—CH₂—OH, —(CH₂)_{c}—CO₂H, —(CH₂)_{c}—SO₃₋, —(CH₂)_{c}—CONH—Bm, —CH₂—(CH₂OCH₂)_{b}—CH₂—CONH—Bm, —(CH₂)_{c}—NHCO—Bm, —CH₂—(CH₂OCH₂)_{b}—CH₂—NHCO—Bm, —(CH₂)_{c}—OH and —CH₂—(CH₂OCH₂)_{b}—CO₂H,
wherein R′ is selected from the group consisting of H, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO3−, —(CH₂)c-CONH—Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H, and
wherein variable a is 2 to 20; variables b and c, are independently, 1 to 20; and Bm is selected from the group consisting of bioactive peptides containing 2 to 30 amino acid units, proteins, antibody fragments, mono- and oligosaccharides, and bioactive drug compounds.

25. The probe of claim 20 having a chemical structure comprising: 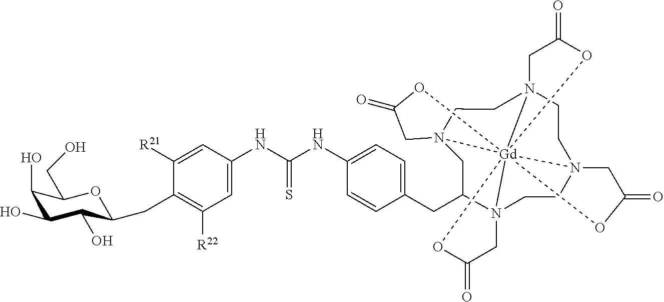
wherein R²¹ and R²² are independently selected from the group consisting of H, CH₂F, CHF₂, CH₂OCONHR′, CH₂OCON(CH₂)ₐ, CH₂OCOOR′, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)_{b}—CH₂—OH, —(CH₂)_{c}—CO₂H, —(CH₂)_{c}—SO₃₋, —(CH₂)_{c}—CONH—Bm, —CH₂—(CH₂OCH₂)_{b}—CH₂—CONH—Bm, —(CH₂)_{c}—NHCO—Bm, —CH₂ (CH₂OCH₂)b-CH₂—NHCO—Bm, —(CH₂)c-OH and —CH₂—(CH₂OCH₂)b-CO₂H;
wherein R′ is selected from the group consisting of H, C1-C10 alkyl, C5-C20 aryl, C1-C10 alkoxyl, C1-C10 polyalkoxyalkyl, C1-C20 polyhydroxyalkyl, C5-C20 polyhydroxyaryl, C1-C10 aminoalkyl, cyano, nitro, halogens, saccharides, peptides, —CH₂(CH₂OCH₂)b-CH₂—OH, —(CH₂)c-CO₂H, —(CH₂)c-SO3−, —(CH₂)c-CONH-Bm, —CH₂—(CH₂OCH₂)b-CH₂—CONH—Bm, —(CH₂)c-NHCO—Bm, —CH₂—(CH₂OCH₂)b-CH₂—NHCO—Bm, —(CH₂)c-OH and —CH₂—(CH₂OCH₂)b-CO₂H, and
wherein variable a is 2 to 20; variables b and c, are, independently, 1 to 20; and Bm is selected from the group consisting of bioactive peptides containing 2 to 30 amino acid units, proteins, antibody fragments, mono- and oligosaccharides, and bioactive drug compounds.

26. A method for detecting or visualizing senescent cells comprising delivering the probes from any of claims 1-25 into one or more cells and using an imaging modality to detect or visualize the senescent cells.

27. The method of claim 26, wherein the cells are either in cell culture or in vivo in a living subject.

28. The method of claim 26, wherein the imaging modality comprises fluorescence imaging, positron emission tomography (PET) imaging, single photon emission computed tomography (SPECT), or magnetic resonance imaging (MRI).

29. The method of claim 27, wherein the living subject is a mammal.

30. The method of claim 29, wherein the living subject is a human, dog, cat, cow, horse, rat, Guinea pig, rabbit, or mouse.
